(19) United States
(12) Patent Application Publication
(10) Pub. No.: US 2004/0189297 A1
(43) Pub. Date: Sep. 30, 2004

(54) Imaging arrangement and process for locally-resolved imaging

(76) Inventors: Michael Bock, Heidelberg (DE); Christian Fink, Heidelberg (DE); Bernd Misselwitz, Glienicke (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/734,585

(22) Filed: Dec. 15, 2003

Related U.S. Application Data
(60) Provisional application No. 60/446,563, filed on Feb. 12, 2003.

(30) Foreign Application Priority Data
Dec. 13, 2002 (DE) 102 603 72.3

(51) Int. Cl. G01V 3/00; A61B 5/05

(52) U.S. Cl. 324/307; 600/419; 600/420; 324/318

(57) ABSTRACT

For easy detection and imaging of even microscopically small pathological structures bordering blood vessels in a human or animal body, especially of lymphatic tissue and arteriosclerotic deposits in the blood vessels, an arrangement and a process are proposed according to which a nuclear spin tomography device is used to obtain data for locally-resolved imaging of the magnetic resonance behavior of the atomic nuclei in a selected field of view in the body, the device being made and programmed such that the body can be exposed by the device to high frequency and magnetic field gradient echo pulse sequences that produce magnetization in the body so that magnetization of a medium that is flowing in at least one direction in space in the body can be attenuated by dephasing the spins of the atomic nuclei in the medium, an MR contrast medium being supplied to the body.

This application claims the benefit of the filing date of U.S. Provisional Application Serial No. 60/446,563 filed Feb. 12, 2003.

### DESCRIPTION

The invention relates to an imaging arrangement and a process for locally-resolved imaging, especially of human or animal bodies. The arrangement and the process are especially suited for magnetic resonance (MR) tomography on the human and animal body.

Lymph vessels in mammal and human bodies were imaged in the past using x-ray processes with direct puncture of the lymph vessels and the lymph nodes with simultaneous administration of x-ray contrast media (direct lymphography). These interventions are very painful to the patient and often lead to side effects.

To image lymphatic tissue, instead of x-ray diagnostics, magnetic resonance tomography can also be used. This technique, like x-ray technology, is likewise well suited for imaging of lymph vessels and the lymph nodes as a result of a multiplanar slice guidance and the high contrast of soft parts. In “Interstitial MR Lymphography with a Conventional Extracellular Gadolinium-based Agent: Assessment in Rabbits”; S. G. Ruehm, C. Corot, J. F. Debatin; Radiology, 2001; 218:664-669, subcutaneous administration of gadoterate-meglumine in rabbits for imaging of the lymphatic system is described.

In experimental studies, good differentiation of metastases in the lymphatic system and of healthy lymph tissue could be demonstrated with novel contrast media.

Depending on the contrast media used, the following disadvantages are indicated:

(a) If the contrast medium is administered interstitially, only part of the lymphatic system can be imaged.

(b) Experimental (not approved) iron-containing contrast media exhibit very slow take-up into the lymph nodes. For this reason, it is necessary to summon the patient to be examined twice, specifically once to administer the contrast medium, and once to carry out the examination. Summoning the patient twice is, however, often impracticable in routine clinical practice.

Moreover, with these contrast media, unfavorable contrast properties arise, specifically negative contrast (signal drop in the target organ), and susceptibility artefacts. For example, very small superparamagnetic iron oxide particles (USPIO) that are coated with dextran can be used. The particles are introduced into functional lymph node tissue in phagocytes, but not into metastatic tissue, in which phagocytosis does not take place. A maximum of accumulation of this contrast medium within the lymph nodes is achieved only roughly 24-48 hours after administration. The target structures are dark, since these substances as negative contrast media clearly reduce the spin-spin relaxation time T₂ and especially T₂* by their susceptibility effect.

(c) In systemic administration of lymphotropic contrast media, the long dwell time that develops there in the blood (intravascular contrast medium) leads to poor distinguishability of the lymph nodes that are ordinarily located directly next to the blood vessels. Examples of these contrast media are given in: “Magnetic Resonance in Medicine,” P. A. Rinck, 4th Edition, Blackwell Wissenschafts-Verlag, Berlin, 2001.

The imaging of arteriosclerotic deposits in the vessel wall, so-called plaques, is also of special interest. Since most intravascular contrast media cause a signal rise within the blood vessels, directly after administering the contrast medium it is almost impossible to distinguish the plaque in the vessel wall. The arteriosclerotic deposits can only be distinguished with great difficulty from the blood vessels immediately after administration to the blood system. Therefore, as for imaging of lymph nodes, it is necessary to allow a long waiting time between administration of the contrast medium and imaging with the imaging hardware. To image plaques, special contrast media can be used. To differentiate the plaques from the interior of the blood vessel, the fact can be used that the contrast medium remains longer in the plaques longer than in the bloodstream so that after a waiting time of roughly 12-48 hours, the plaques are still signal-amplified, but the blood is again imaged with a weaker signal. As for lymphography, for the imaging of plaques in everyday clinical practice, it is, however, often not feasible to observe such a long waiting time between administration and recording, since a patient would then have to be summoned twice for the examination.

In the examination of a body, especially a human or animal body, with a nuclear resonance experiment either a so-called spin-echo sequence with 90°-180° excitation or instead of or in addition to this excitation a measurable signal sequence can also be achieved with a gradient pulse sequence (gradient echo method). After the 90° high frequency pulse in this case, first a gradient pulse is switched, for example, in x-direction. Afterwards, a gradient pulse is applied in x-direction, i.e., with an inverted gradient sign. In this way, the initially dephased spins are refocussed into a measurable signal that can be imaged in a coordinate system rotating with ω₀ as the sum vector.

For locally-resolved imaging of the nuclear spins in a field of view (FOV) to be examined in a body, the nuclear spins must be assignable to individual space elements. Here, the effect that the Larmor frequency ω(x,t) is a function of the magnetic field intensity B(x,t) is used. In slice scanning when using a magnetic field gradient in the form of a gradient pulse, during the 90° pulse in z-direction only one thin layer (“slice”) is excited, in which the magnetic field intensity B₀ corresponds specifically to the Larmor frequency ω₀. Because only cores are excited that are located within the slice under resonant conditions, simple assignment of cores is achieved, for example, in z-direction. In order to also achieve local resolution in x- and y-direction, after using the 90° pulse, other gradient pulses are switched along the x- or y-direction: In y-direction gradient switching is inserted following the 90° pulse (“phase”). The local information in y-direction is contained in the phase shift of the preceding nuclear spins that is caused by this temporarily switched gradient (phase coding). The location information is made accessible from the response signal by Fourier transformation (FT) analysis. In order to achieve different phase shifts in y-direction and thus to obtain unique location information of the nuclear spins in this direction in space, gradient switchings in succeeding pulse sequences are inserted that are raised or lowered in succession in increments, the phase gradients being varied with the gradient slopes between the two maximum values +G and −G. In the same way, in x-direction, a first gradient switching is also inserted (“read”) and it contains the required information about the local resolution of the nuclear spins in x-direction. To generate an echo signal, a second read-gradient switching with a different polarity from the first is then inserted, which, after dephasing the spin in the x-y plane based on the first read-gradient switching, leads to refocussing the nuclear spins as a result of the switching of the second pulse so that a response signal is formed. Since, due to the read-gradient switching depending on the location in x-direction, different magnetic fields in the region B₀±ΔB are present during refocussing, the signals originating from the different locations can be separated using different frequencies in the range ω₀±Δω (frequency coding). In turn, FT analysis is used for local imaging.

The measurement can be accelerated by the nuclear spins being excited with an RF pulse that leads to tilting of net magnetization by less than 90° (flip angle α<90°).

While the individual slices in the body to be examined are examined in succession in the above-described 2D-FT, three-dimensional imaging of a body to be examined can also be produced in a single pulse sequence without slicing (3D-FT): To do this the aforementioned pulse sequences are used for the phase- and readout-gradient pulse. The slice-gradient pulse during the RF pulse is followed in addition by a downstream slice-gradient pulse with inverted polarity, the second slice-gradient pulse in succeeding pulse sequences being raised or lowered in increments between two maximum values +G and −G.

To image blood vessels (angiography), various techniques have been used; in some cases they consist in suppressing the signal from the blood vessels in a recording sequence and recording it in another sequence with flow compensation, i.e. without dephasing of the moving nuclear spins (signal carrier). To differentiate the vessels from the surrounding quiet tissue, a difference between the two recordings is found that produces good contrast between the vessels and the surrounding tissue, the blood vessels being imaged brightly. A comparison of the process is contained in “Black Blood Angiography,” W. Lin, M. Haacke, R. R. Edelman; in “Magnetic Angiography, Concepts and Applications” (Editors: E. J. Potchen, E. M. Haacke, J. E. Siebert, A. Gottschalk), Mosby, St. Louis (1993).

Since the beginning of clinical MR imaging, processes have been used with which MR signals of moving signal carriers can be suppressed. Important processes for suppressing moving MR signal carriers will be discussed below.

a) In the standard MR measurement sequence, the spin-echo sequence, moving spins are intrinsically suppressed since those spins that leave the measurement slice between the 90° excitation pulse and the 180° refocussing pulse do not contribute to the MR signal. This propagation time effect becomes stronger with increasing echo time TE that is twice as long in a standard spin echo sequence with 90° and 180° pulses as the time interval between the 90° pulse and the 180° pulse, or reduced slice thickness. This technique, however, is not suited for gradient echo sequences without 180° pulses. Therefore, this effect cannot be used for a fast recording time: In any case it is technically almost impossible to image three-dimensional volumes with spin-echo sequences in practicable measurement times, while this is done in a few seconds with high speed gradient echo processes, especially with magnetic resonance angiography amplified by means of contrast media.

b) In conventional magnetic resonance processes for suppressing blood vessel signals, the signal of the blood vessels is saturated outside the imaging slice by for example so-called saturation slices being positioned parallel to the measurement slice. Since 180° pulses are not used in signal readout here, this saturation process can be combined with almost any imaging technique in magnetic resonance technology. Basically, with this process the advantage is used that blood compared to other tissues has a very long Ti relaxation time and in the signal readout that follows directly on saturation, only saturated blood is present in the measurement slice and delivers almost no signal. In a variant of this process the magnetization is inverted only outside the measurement slice. Afterwards, it is awaited until the lengthwise magnetization of the signal carrier in the blood (along the z-axis) has a zero passage as a result of T, relaxation. The signal carriers that have flowed into the measurement slice in the blood then do not contribute to the MR signal.

c) In gradient echo images, it was observed relatively early that blood flowing quickly from a vascular constriction in a certain swirl zone (jet) causes artificial signal reduction. This effect is based on the fact that the moving spins under the action of gradients that are needed for location coding in MR imaging accumulate an additional phase that is dependent on the velocity of the spins. In the jet, within each pixel, many different phases occur so that phase-coherent addition of the MR signals leads to a reduction of the cumulative signal in the MR image. This phenomenon is called intravoxel incoherent motion and is also known from diffusion-weighted MR imaging, This effect can be intensified by the gradients being added to the imaging such that the location coding remains unaffected, while the velocity-dependent phase is maximized. This technique is called black blood angiography. It is implemented in conjunction with spin echo sequences in order to use their additional signal suppression. Conversely, this effect can also be used for imaging of blood vessels by subtracting two data sets with and without additional gradient switching from one another (rephase/dephase imaging).

For imaging of blood vessels in clinical practice, either the spin echo method that is made more efficient by the built-in dephasing gradients (black blood angiography) is used, or the rephase-dephase method that is used mainly for positive imaging of the peripheral arteries is used. Neither technique has been used to date in combination with contrast media. Rather, here the intrinsic contrast of the moving blood acts to image the blood vessels. In the rephase/dephase method this means that in the subtraction of the two data records, vessels only appear bright when enough fresh blood is flowing into the measurement slice.

In any case, studies by means of black blood angiography are also known that have been used, for example, to image arteriosclerotic deposits in blood vessels. Thus, in “Extracranial Carotid Arteries: Evaluation with “Black Blood MR Angiography”, R. R. Edelman, H. P. Mattle, B. Wallner, R. Bajakian, J. Kleefield, B. Kent, J. J. Skillman, J. B. Mendel, D. J. Atkinson: Radiology: 1990; 177:45-50, a comparison of bright blood angiography to black blood angiography for imaging of pathological changes of the carotid artery is described. Black blood angiography should offer the advantage over bright blood angiography that dysfunctions can be imaged very accurately. To image lesions in black blood angiography, a 2D-spin-echo method was used, since gradient echo sequences were not suitable for suppression of the moving nuclear spins, although the examined slices were saturated. To achieve suppression, the echo time TE would have had to be prolonged. This, however, would have led to a reduction in the resolution of the structures on the blood vessels and to reduced contrast between the blood vessel and muscle tissue.

With previously known processes for suppression of blood vessels, it has been fundamentally possible to avoid their imaging when there is no contrast medium in the blood flow. If, however, a contrast medium that shortens the relaxation time is added, in the imaged slices in a human or animal body it is almost impossible to easily recognize pathological structures in the lymph vessels and arteriosclerotic deposits in the blood vessels, especially when the latter are relatively small.

Therefore, the object of this invention is to find means with which especially small pathological structures can be easily recognized and imaged. Mainly high-contrast, distinct imaging, free of superposition, of stationary structures that adjoin the blood vessels in the human or animal body will be possible. In particular, metastases in lymph tissue and in plaques will be easily and quickly recognized and imaged.

The problem is solved by the imaging arrangement indicated in claim 1 and the process indicated in claim 9. Advantageous embodiments of the invention are given in the subclaims.

If below and in the claims it is indicated that magnetic field gradient echo pulse sequences are switched in a certain direction in space, it is to be understood that the sequences can be switched in one or two optional or all three directions in space. In the same way, with the indication that magnetization of the flowing medium can be attenuated in one direction in space, it is to be understood that magnetization can be attenuated in one or two optional directions in space or in all three directions in space.

To image pathological structures with in part microscopically small dimensions in the lymphatic system and in the blood vessels by means of magnetic resonance tomography, a magnetic resonance (MR) contrast medium is used that is taken up into the body that is to be examined. For imaging purposes, a nuclear spin tomography device is used to obtain data for locally-resolved imaging of the magnetic resonance behavior of the atomic nuclei in a selected field of view in a body. The device is made and programmed for this purpose such that the body is exposed by the device to high frequency and magnetic field gradient echo pulse sequences that produce magnetization in the body. The magnetization of signal carriers (spins of atomic nuclei, especially ¹H nuclei) that are located in a medium that is flowing in at least one direction in space, especially blood, is attenuated by dephasing the spins of the atomic nuclei in this flowing medium so that imaging of structures located in the immediate vicinity of the flowing medium is greatly simplified, even if they are microscopically small, since blood is imaged dark in this way. Only by administering an MR contrast medium is it possible to find the desired fine structures specifically and to recognize them with certainty.

By implementing the invention, for example, lymph nodes of a certain region in the human or animal body or the entire body can be imaged with high spatial resolution, since, on the one hand, the moving signal carriers from the blood vessels are suppressed and the target structures, on the other hand, are displayed intensified by the contrast media, so that they are especially well emphasized. The signal intensity originating from the blood vessels is selectively suppressed according to the invention so that for example the lymph nodes in the immediate vicinity to large blood vessels can be imaged and distinguished from the vessel. The same applies to arteriosclerotic deposits, so-called plaques, in the blood vessel walls.

Conventional saturation processes that have been developed to suppress moving signal carriers conversely cannot be used to distinguish the lymph nodes and plaques in combination with contrast media, since the saturated magnetization in the presence of the contrast medium recovers within a few milliseconds and is available in the following signal readout. This effect is caused by the massive T₁ shortening that is dependent on the contrast medium concentration. In contrast to this, the dephasing of the nuclear spins according to the invention can be easily achieved by using gradient pulse sequences in the presence of contrast media and is therefore superior to it. Compared to the rephase/dephase imaging method, the process according to the invention is roughly twice as fast since the rephase part of the method can be abandoned.

To image in particular lymphatic tissue and arteriosclerotic deposits in blood vessels, MR contrast media are used that are advantageously tailored if necessary to the respective application. The contrast media should preferably meet the following conditions:

a) They should lead to signal amplification in the MR image with the selected sequence.

b) They should accumulate in the target structure, i.e., in lymphatic tissue or in the arteriosclerotic deposits. To do this, it is, of course, necessary for the contrast media for imaging of lymph nodes to be lymph-passable and for imaging of plaque to be plaque-passable.

c) They should also accumulate in the blood vessel system.

To detect metastases of the lymphatic system, for example, the already aforementioned coated iron oxide particles in the form of USPIO are suitable. In any case, the coated iron oxide particles require a longer time for concentration in the lymph nodes. Moreover, these contrast media are not suited for imaging of the lymph vessels due to the negative contrast.

Among others, mainly gadolinium-containing compounds can be used advantageously. For lymphography, it is possible to use polymer compounds, like the compounds described by L. Harika, R. Weissleder, K. Poss, C. Zimmer, M. I. Papisov, T. J. Brady in “MR Lymphography with a Lymphotropic T₁-Type MR Contrast Agent: Gd-DTPA-PGM”; MRM; 1995; 33:88-92 and by G. Staatz, C. C. Nolte-Ernsting, A. Bucker et al. in “Interstitial T₁-Weighted MR Lymphography with Use of the Dendritic Contrast Agent Gadomer-17 in Pigs”; Rofo. Fortschr. Geb. Röntgenstr. Neuen Bildgeb. Verfahr.; 2001; 173:1131-1136, and lipophilic compounds that form aggregates or micelles like the compounds described by B. Misselwitz, J. Platzek, B. Raduechel, J. J. Oellinger, H. J. Weiumann in: “Gadofluorine 8: Initial Experience with a New Contrast Medium for Interstitial MR Lymphography”; Magma; 1999; 8:190-195 and by G. Staatz, C. C. Nolte-Ernsting, G. B. Adam et al. in “Interstitial T₁-Weighted MR Lymphography: Lipophilic Perfluorinated Gadolinium Chelates in Pigs”; Radiology, 2001; 220:129-134.

Those compounds are especially suitable that are already accumulating in the lymphatic tissue within a very short time after administration. They are preferably gadolinium complexes that are provided with polar radicals, for example sugar radicals, and fluorinated side chains and that are aggregated into micelles with a size of 4-6 nm. Such compounds are described in, for example, WO 02/14309 A1. With these contrast media, MR examination can already be carried out within a few minutes up to one hour after administration. These special gadolinium compounds can also be used to image arteriosclerotic deposits (plaques).

Furthermore, compounds of other paramagnetic metal ions can be used, for example compounds of Mn(II), Dy(III) and Fe(III). Gd(III), Mn(II) and Fe(III) compounds act as positive contrast media since these media reduce the longitudinal relaxation time T₁ so that those parts are brightened in an MR image into which the contrast medium has been absorbed. Conversely, Dy(III) compounds as well as iron oxide particles act as negative contrast media since they reduce T₂ and especially T₂* due their susceptibility effect, so that the parts appear darker in an MR image into which these contrast media have been absorbed. In this respect, the latter compounds are not as well suited as Mn(II) and Fe(III) compounds.

Instead of the aforementioned contrast media, other types of contrast media can also be used, for example nitrogen oxides that like the aforementioned metal ions are paramagnetic. Furthermore, gas-filled microbubbles are proposed that can be filled, for example, with nitrogen or perfluoropropane. Such systems are described in, for example, U.S. Pat. No. 6,315,981 A.

Furthermore, instead of paramagnetic or superparamagnetic substances, diamagnetic compounds can also be used as contrast media; they do not contain ¹H, but rather other signal carriers, for example fluorocarbon compounds. Instead of ¹H MR tomography, in this case ¹⁹F-MR tomography is carried out since the ¹⁹F atomic nucleus also has a nuclear spin of ½, the gyromagnetic ratio for ¹⁹F being distinctly different from that for ¹H so that these atomic nuclei in the MR image form an image contrast. They should be compounds that are taken up into the target structures. If these compounds have a long dwell time in the blood, the target structures can be made selectively visible with this invention without the blood vessels preventing recognition of these structures.

The MR contrast medium can be administered especially intravenously to a human or animal body. The contrast medium, however, can also be administered intraarterially, percutaneously, especially subcutaneously, furthermore perorally, intraperitoneally, intramuscularly or in some other way.

To attenuate the magnetic resonance signals from the spins in the flowing medium, according to the invention the effect is used that the spins of the atomic nuclei contained in the field of view in the body to be examined dephase during motion, while this does not apply to stationary spins. This can be achieved by suitable switching of the magnetic field gradient pulses. In order to determine under which conditions the signals are attenuated, the following equation for the phase of the respective nuclear spins is assumed; it is dependent on location and time and it is a function of the location x within a gradient field, the time-dependent gradient field intensity G(t) and the time t after excitation of the atomic nuclei with a high frequency pulse:

$$ϕ ( x , t ) = γ \cdot {\int }_{0}^{t} G ( {t}^{′} ) \cdot x ⅆ {t}^{′} \qquad [ 1 ]$$

The constant γ is the gyromagnetic ratio, and for the protons that are primarily used in magnetic resonance imaging, in practical units it is 2π42.577 MHz/T.

If at this point the excited atomic nuclei are moving while a gradient is being turned on, with a component of motion parallel to the spatial direction of the gradient, the location x at which the atomic nuclei are located at time t is likewise a function of time. Therefore, equation [1] can be reformulated as follows:

$$ϕ ( t ) = γ \cdot {\int }_{0}^{t} G ( {t}^{′} ) \cdot x ( {t}^{′} ) ⅆ {t}^{′}$$

By expansion into a Taylor series and ignoring higher terms, this yields the following relation:

$$\begin{matrix}ϕ ( t ) = γ \cdot {\int }_{0}^{t} G ( {t}^{′} ) \cdot ( {x}_{0} + {v}_{0} \cdot {t}^{′} + \frac{{a}_{0}}{2} \cdot t + … ) ⅆ {t}^{′} \\ \approx γ \cdot {x}_{0} \cdot {\int }_{0}^{t} G ( {t}^{′} ) ⅆ {t}^{′} + γ \cdot {v}_{0} \cdot {\int }_{0}^{t} G ( {t}^{′} ) \cdot {t}^{′} ⅆ {t}^{′}\end{matrix} \qquad [ 2 ]$$

x₀ is the origin of the atomic nucleus in motion during a gradient pulse sequence, and v₀ is the constant speed of the flowing medium. As the abbreviation for the time integrals, multiplied by γ, M₀ and M₁ are introduced so that the following relation is produced:

ρ(t)=x₀·M₀+v₀·M₁ [2a]

M₀ is known as the gradient moment of the order zero and M₁ as the gradient moment of first order. Time-dependent gradient moments of order i Mᵢ(t) that have already been ignored in equation [2a] are defined as follows:

$${M}_{i} ( t ) = γ \cdot {\int }_{0}^{t} G ( {t}^{′} ) {t}^{′ i} ⅆ {t}^{′} \qquad [ 3 ]$$

Gradient switchings, in which M₀ (gradient moment of order zero) is zero, are necessary to generate echo signals produced by gradient switchings, since the nuclear spins rephase only under this condition. Conventional gradient switchings in which M₁ (gradient moment of first order) is zero are called flow-compensated, since here the nuclear spins that move with a constant velocity in a flowing medium do not experience the additional dephasing that is caused by the motion, so that they appear bright in imaging. The definition of the gradient moments Mᵢ results only in the low moments contributing to the signal phase for short times, while higher moments scale with t¹ and thus remain small.

The statements above indicate that M¹ must be as large as possible to suppress the signals from the moving nuclear spins since the dephasing is especially great in this case. This means that the magnetic resonance signals of the medium flowing in at least one direction in space in the body can be attenuated by flow dephasing gradient pulses by a gradient moment of the first order M₁(t) being maximized in this direction in space according to the following relation:

$${M}_{1} ( t ) = γ \cdot {\int }_{0}^{t} G ( {t}^{′} ) \cdot {t}^{′ } ⅆ {t}^{′}$$

whereby

γ is the gyromagnetic ratio of the atomic nuclei,

G(t′) is a time-dependent gradient field intensity in this direction in space and

t is the time interval that has passed since the injection of a high frequency pulse for excitation of the atomic nuclei.

By taking into account gradient moments of higher order according to equation [3] with i>1, dephasing of flowing media that have not only a constant speed, but are also accelerated or slowed down during the gradient switchings, can be achieved.

In a preferred embodiment of the invention, magnetization of the medium flowing in at least one direction in space in the body is attenuated by dephasing of the spins such that the gradient moments of order i Mᵢ(t), especially gradient moments of the first order M₁(t), are maximized in this direction in space.

If, for example, within one pixel there are atomic nuclei with velocities within a velocity interval from 0 to vₘₐₓ with the same frequency and the pertinent nuclear spins are imaged with the same signal intensity, the cumulative signal of these nuclear spins disappears exactly when the phase caused by M₁ is exactly 2π (i.e. 360°) so that the following relation applies:

2π=νₘₐₓ·M₁ [4]

vₘₐₓ being the maximum velocity of the flowing medium in the body that is to be examined, up to which dephasing is not effectively achieved.

For velocities that are greater than vₘₐₓ, the cumulative signal remains very small, so that vₘₐₓ can be interpreted as the boundary velocity below which signal suppression in the flowing medium does not work effectively. This means that the signal of moving nuclear spins is preserved and is not suppressed when these nuclear spins are moving with a velocity that is less than vₘₐₓ. Therefore, knowledge of the typical velocities in the blood vessels to be examined is of interest to be able to effectively use the invention. Since blood in venous structures moves only very slowly, these structures can in general be easily recognized since the nuclear spins obtained there are not dephased and thus suppressed. This is however not a disadvantage for imaging of lymphatic tissue and plaques, since the latter are adjacent rather to the arteries. If venous flow is also to be suppressed, stronger and/or longer gradient switchings must be used.

Generally a given gradient switching will have a non-disappearing gradient moment of the first order M₁, so that the gradient switching is not flow-compensated. Such gradient switchings are conventionally used for receiving stationary signal carriers. In any case, to suppress a slow flow, gradient moments that are not achieved by typical imaging gradients are necessary.

Because special gradient switchings are used that lead to flow dephasing, in an embodiment according to the invention, conventional imaging 2D- or 3D-gradient echo pulse sequences, especially flow-compensated gradient echo pulse sequences, into which flow dephasing gradient pulses are inserted, can be used.

If the gradients are modified in an existing gradient echo pulse sequence that is used for imaging, for example in a flow-compensated gradient echo pulse sequence such that a large gradient moment of the first order M₁ is formed, then a new sequence, the flow dephasing gradient pulse sequence that meets the condition according to equation [4] and that thus leads to maximization of M₁ can be added to the existing imaging gradient echo pulse sequence. This condition is necessary so that space coding of the magnetic resonance signals remains unaffected such that the following relation is satisfied:

$${M}_{0} = {\int }_{0}^{t} G ( {t}^{′} ) ⅆ {t}^{′} = 0 \qquad [ 5 ]$$

This condition can be clearly interpreted in that +M₀ then specifically represents the area under the gradient-time curve. One simple possibility for satisfying this relation is to use bipolar gradient pulses, i.e. two gradients of different polarity, whereby their respective intensity and length can be different, but the pulses can especially also have the same intensity and length.

For example, the nuclear spins in one direction in space can be dephased by switching the gradient pulse with a time integral A by a certain amount. By later switching of a second gradient pulse with the time integral-A in the same direction in space, the stationary signal carriers are again completely rephased, but moving signal carriers are not.

Alternatively to the variant in which the flow dephasing gradient pulses are added to the flow-compensated gradient echo pulse sequence, in another embodiment of the invention a non flow-compensated gradient echo pulse sequence can also be assumed. After adding the additional pulse sequence, however, the aforementioned conditions must be met, according to which M₀=0 (equation [5]) and M₁ according to equation [4] is maximized.

When using contrast media for better imaging of microscopically small structures in lymph nodes or of arteriosclerotic deposits, the selected pulse times for the gradient moments must be very small, since the relaxation times are very short due to use of the contrast media. When using very short gradient pulses, however, a correspondingly high gradient field intensity can be switched in the short time interval that is available. In the implementation of motion-sensitive gradient pulses, therefore, in addition the following technical boundary conditions must be watched: To produce gradient pulses, gradient systems are used that consist of current-carrying coils. These coils are driven by a current amplifier. These amplifiers can deliver only a finite power, so that the amount of gradient field intensity is limited in practice. Currently, the gradient field intensity in clinical magnetic resonance tomographs is limited, for example, to 30-40 mT/m:

|G(t)|≦Gₘₐₓ [6a]

This value may, however, be higher in the future.

Since the coil turns of the gradient system represent an inductance, according to Lenz's law, moreover, a minimum time is needed to switch to the maximum gradient field intensity. This minimum time interval is, of course, like the maximum gradient field intensity, dependent on the respective technical possibilities such that a reduction of the required time is a function of technical progress. The rise time is generally given in the form of a slew rate sₘₐₓ

$$\vert s ( t ) \vert = \vert \frac{ⅆ G ( t )}{ⅆ t} \vert \leq \frac{{G}_{max}}{{t}_{min}} = {s}_{max} \qquad [ 6 b ]$$

The aforementioned conditions according to equations [6a] and [6b] can be easily implemented, for example, by a flow dephasing gradient pulse sequence with long pulses being used. For example, the gradient moment of first order M₁ for a bipolar gradient pulse can be given by

M₁(t; G_{bipolar}, tᵣₐₘₚ, tₚₗₐₜₑₐᵤ, tₛₑₚ)=γ·G_{bipolar}·(tᵣₐₘₚ+tₚₗₐₜₑₐᵤ)·(2ₜᵣₐₘₚ+tₚₗₐₜₑₐᵤ+tₛₑₚ₎ [7]

whereby

G_{bipolar} is the maximum gradient field intensity,

Tᵣₐₘₚ is the rise/fall time when the gradient field is turned on/off,

Tₚₗₐₜₑₐᵤ is the time interval during which G_{bipolar} is reached, and

tₛₑₚ is the time interval between two gradient pulses.

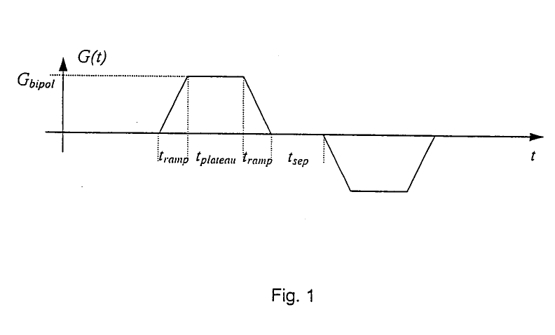

Reference is made to FIG. 1 for a more detailed explanation of these parameters.

For contrast medium-supported examinations according to the invention, the gradient pulses for flow dephasing must be kept as short as possible. In particular, the gradient pulses used additionally for flow dephasing should be as short as possible, since a shortening of the longitudinal relaxation time T₁ caused by the contrast medium inevitably also accompanies a shortening of the transversal relaxation time T₂. If under these conditions long gradient pulses were switched, the echo times TE for signal readout would be prolonged so that as a result, a stronger signal loss both for moving and for resting signal carriers would result due to the accelerated T₂ decay.

In a preferred embodiment of the invention, the gradient pulse sequence comprises flow dephasing gradient pulses in the three directions in space (orthogonally on one another in the Cartesian coordinate system). The gradient echo pulse sequences in the respective directions in space are formed in the case when the flow dephasing gradient pulses are inserted into the imaging gradient echo pulse sequences.

Of course, flow dephasing gradient pulses can also be inserted into imaging gradient echo pulse sequences in only one or only two directions in space. This can be advantageous, for example, when the flowing medium is not to be attenuated in the directions in space in which the flow dephasing gradient pulses are not inserted. Thus, it can be of interest especially to suppress the aorta by switching flow dephasing gradient pulses in z-direction.

Gradient echo pulse sequences can be selected in any manner if only M₀=0 and M₁ are as large as possible, since the exact shape of these gradient pulses is irrelevant to the implementation of this invention. Of course, the time spent for inserting the additional flow dephasing gradient pulses should be short in order to minimize the echo times of the sequence. This is necessary since the signals of all structures that accumulate a contrast medium in a body have a shortened T₂ decay that would lead to massive signal loss for long echo times.

Basically, the invention can be implemented in two embodiments. To do this, two different variants of flow dephasing are used. It is common to the two variants that a gradient echo pulse sequence is switched that meets the conditions according to which M₀=0 (equation [5]) and M₁ according to equation [4] is maximized. In addition, the secondary conditions formulated in equations [6a] and [6b] must be maintained:

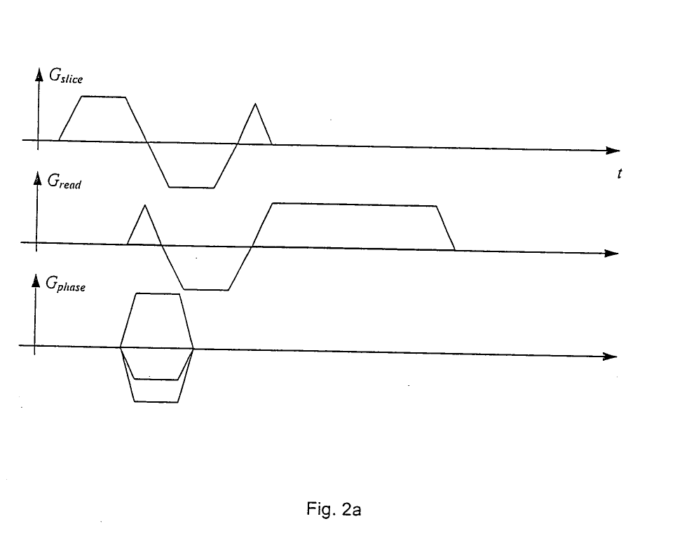

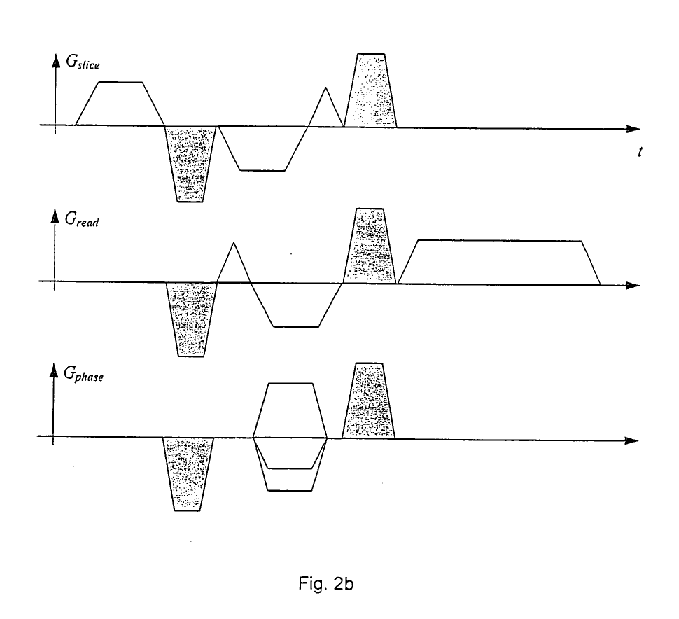

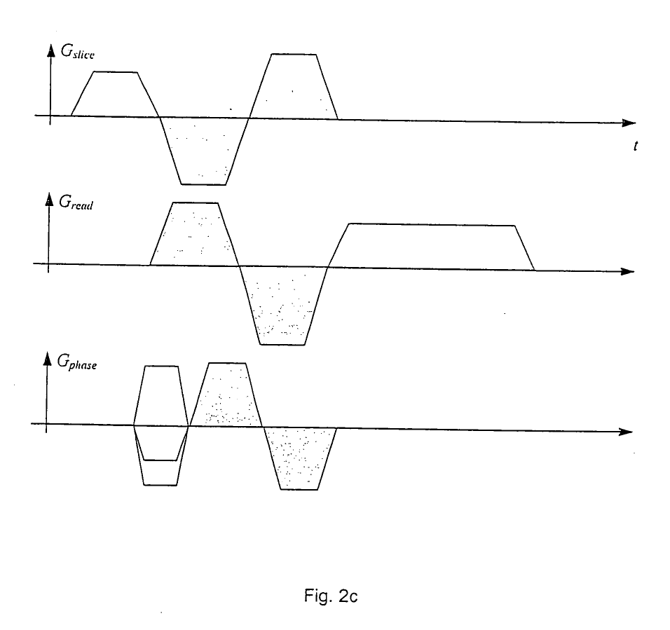

1. In a first implementation, bipolar gradient pulses in the frequency and phase coding direction before signal readout and in the slice selection direction after high frequency excitation are additionally inserted between the actual imaging gradients (see in this respect also FIGS. 2a and 2b). The parameters G_{bipolar}, tᵣₐₘₚ and tₛₑₚ can, for example, be stipulated so that for a minimum plateau time tₚₗₐₜₑₐᵤ=0 ms a maximum value for vₘₐₓ results. In order to also be able to implement gradient moments of the first order M₁ (and thus small vₘₐₓ), the gradient echo pulse sequence can be programmed, for example, such that with increasing echo time TE>TEₘᵢₙ plateau times tₚₗₐₜₑₐᵤ are symmetrically added according to

$${t}_{plateau} = \frac{1}{2} \cdot ( TE - {TE}_{min} ) \qquad [ 8 ]$$

Thus, the gradient moment of the first order M₁ according to equation [7] and the velocity Vₘₐₓ above which massive suppression of signals can be expected can be set indirectly via the echo time TE according to equation [4].

2. In an optimized implementation, all the imaging gradients of a given pulse sequence that are used between the high frequency excitation and signal readout are recomputed such that the additional gradient contributions to maximizing the gradient moment of the first order M₁ implement a gradient moment of the first order M₁ that is given via a boundary velocity vₘₐₓ and at the same time do not change the gradient moment of order zero M₀ of the original gradient train (see FIG. 1c in this respect). Here, it is often necessary to prolong the echo time TE of the gradient train. The echo trains produced with this implementation are always shorter, however, than the trains described under 1., since the imaging and flow dephasing gradient pulses here are played out at the same time and not in succession. The gradient timing that is shortest under the given boundary conditions according to equations [6a] and [6b] is found in one such approach by numerical optimization.

Basically, it applies that the difference between the two processes at large boundary velocities vₘₐₓ of the nuclear spins that can be dephased with relatively short and weak gradient pulses is the greatest, while the flow dephasing gradient pulse sequences compared to the imaging gradient echo pulse sequences at low velocities make a major contribution to gradient timing, such that the echo times differ only slightly.

Thus, basically two processes for suppression of signals in moving media by dephasing of nuclear spins are available, in which the gradient echo pulse sequences to be used comprise flow dephasing gradient pulse sequences in at least one direction in space, gradient echo pulse sequences being formed in the respective direction in space by inserting the respective flow dephasing gradient pulses into the imaging gradient echo pulse sequences or being computed according to the aforementioned boundary conditions. The nuclear spins that are moving in the directions in space in which the flow dephasing gradient pulses are active are dephased by the inserted or recomputed sequences.

Essentially the readout of data for imaging can be configured as desired. One advantageous pulse sequence is the so-called FLASH (Fast Low Angle Shot) sequence in which an excitation pulse is radiated with a flip angle α<90°, for example 25°, and gradient pulses are used for refocussing. Additional gradients are used for imaging and flow dephasing. The time necessary for data acquisition is reduced when the excitation pulse is radiated with a flip angle α<90°.

To accelerate the recording, basically also multipulse sequences can be used, for example EPI (echo planar imaging). In these sequences only one excitation pulse is radiated and a host of gradient pulses are switched in succession for locally-resolved imaging such that refocussing signals are obtained with each readout gradient pulse. Thus, in a gradient echo pulse sequence, data for example for a series or an entire matrix in k-space (the received measurement data before conversion into locally coded image data by Fourier transformation) can be recorded. EPI is advantageous with respect to the fact that the data are scanned rapidly. In this case, however, there is the disadvantage that in pictures of many regions of the body, especially in the abdominal area, artefacts appear that necessitate modifications, for example, segmented EPI.

In a main variant of this invention, the data are recorded point for point with separate gradient echo pulse sequences such that for each point, a new excitation pulse is emitted. This procedure is somewhat more time-consuming that the processes in which multipulse sequences are used. The method is much more robust, however, than a process with multipulse sequences. EPI moreover has the disadvantage that image blurring and signal losses occur with the relatively long echo times when contrast media are used that accelerate T₂* decay.

In an alternative procedure according to the invention, an imaging sequence can also be used in which first transversal magnetization is produced by the spins that are aligned in z-direction first being folded down at least in part by emitting a 90° pulse or a pulse with a flip angle α<90° into the x-y plane, then the spins with a suitable flow dephasing gradient pulse for moving spins at which M₀=0 being dephased and the spins finally being folded back again into z-direction by a second 90° pulse. Dephasing is impressed on the magnetization stored in this way in z-direction as additional contrast such that it can be read out with any imaging sequence. This embodiment compared to the FLASH sequence has the disadvantage, however, that the fast Ti relaxation caused by the contrast media levels evens out the contrast impressed in z-direction again.

Furthermore, it is also conceivable in addition for a 180° pulse to be emitted for refocussing. It is very disadvantageous in this procedure, however, that data acquisition takes a much longer time than in exclusive switching of a readout gradient for refocussing. Moreover, in this way very much more energy is emitted into the body to be examined. This leads to a disadvantageous burden on the object under examination.

For further acceleration of the recording technique, data acquisition can also be reduced in that it is not the maximum amount of data that is recorded in the data matrix that is to be subjected to a Fourier transformation in k-space. For example, in one embodiment, only half the amount of data is recorded and the other half is filled with zeros. In another embodiment, only 80% of the lines in k-space are recorded. The remainder is filled in turn with zeros. In all such cases, limited resolution of the imaging is tolerated. This is adequate in many cases, however, for clinical diagnosis, at least for a first orientation examination.

The device according to the invention has especially the following important features:

a static magnet, especially a superconductive electromagnet,

gradient devices for producing gradient pulses in three directions in space that are orthogonal to one another; these devices are formed by current-carrying coils,

a transmission device for producing high frequency signals, especially here an RF transmission coil,

a receiving device for high frequency signals; in this case, this is preferably an RF receiving coil,

a device for triggering the gradient devices and the transmission device; in this case, these are amplifiers, and programmable devices with which the gradient pulse sequences can be generated; furthermore here also these are programmable devices with which the transmitting and receiving coils can be triggered,

an evaluation device, and

a display device.

The transmitting device and the receiving device can be implemented in a preferred embodiment of the invention by a common device. In this case, there is additionally a changeover switch that is used for triggering these devices and that switches between the transmitting mode and the receiving mode.

For a more detailed explanation of the invention, the following figures that are described within the framework of the individual examples are used. In detail:

FIG. 1 shows a diagrammatic visualization of a gradient echo pulse sequence;

FIG. 2a shows a diagrammatic visualization of a flow-compensated gradient echo pulse sequence for recording two-dimensional MR data without special gradient switchings for suppression of moving MR signal carriers;

FIG. 2b shows a diagrammatic visualization of a gradient echo pulse sequence for recording two-dimensional MR data in a first embodiment according to the invention with the flow dephasing gradient pulses inserted (labeled dark);

FIG. 2c shows a diagrammatic visualization of a gradient echo pulse sequence for recording two-dimensional MR data in a second embodiment according to the invention with recomputed flow dephasing gradient pulses that are used at the same time for imaging and for suppressing moving MR signal carriers;

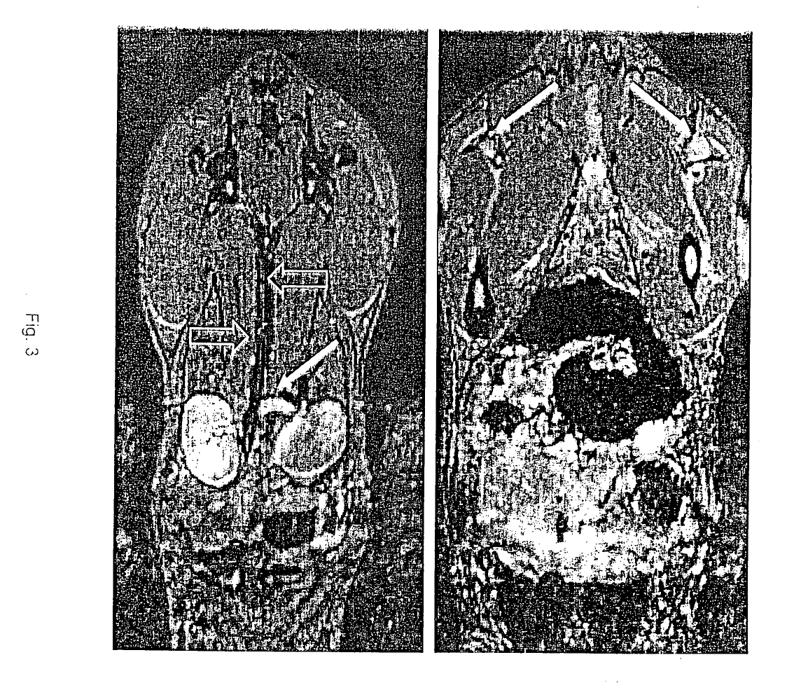

FIG. 3 shows individual pictures, recorded with a 3D gradient echo pulse sequence with flow dephasing in one direction in space, of a Copenhagen rat after administering a lymph-passable MR contrast medium;

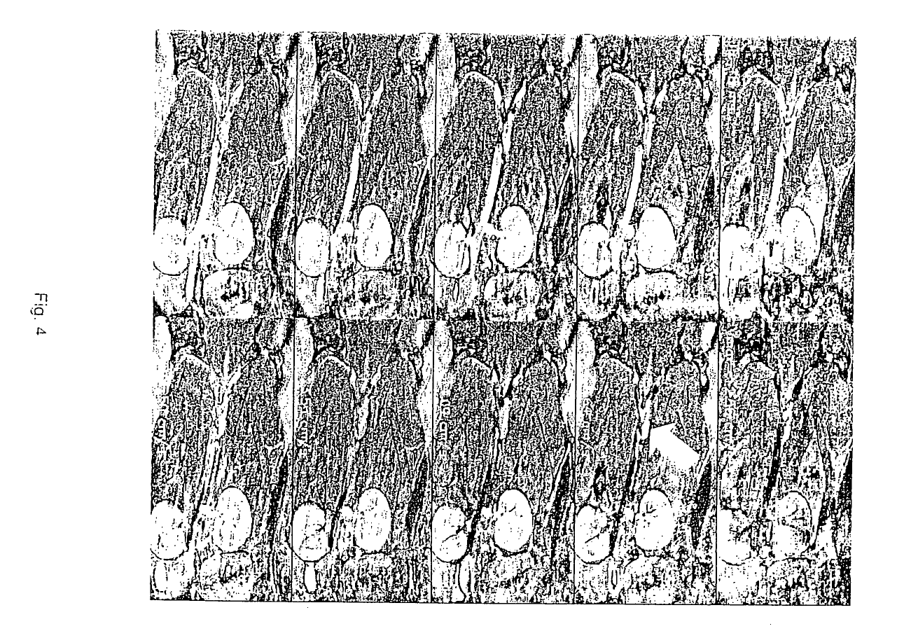

FIG. 4 shows a comparison of individual pictures, recorded with a 3D gradient echo pulse sequence, of a Copenhagen rat, with and without flow dephasing gradient switchings in all three directions in space;

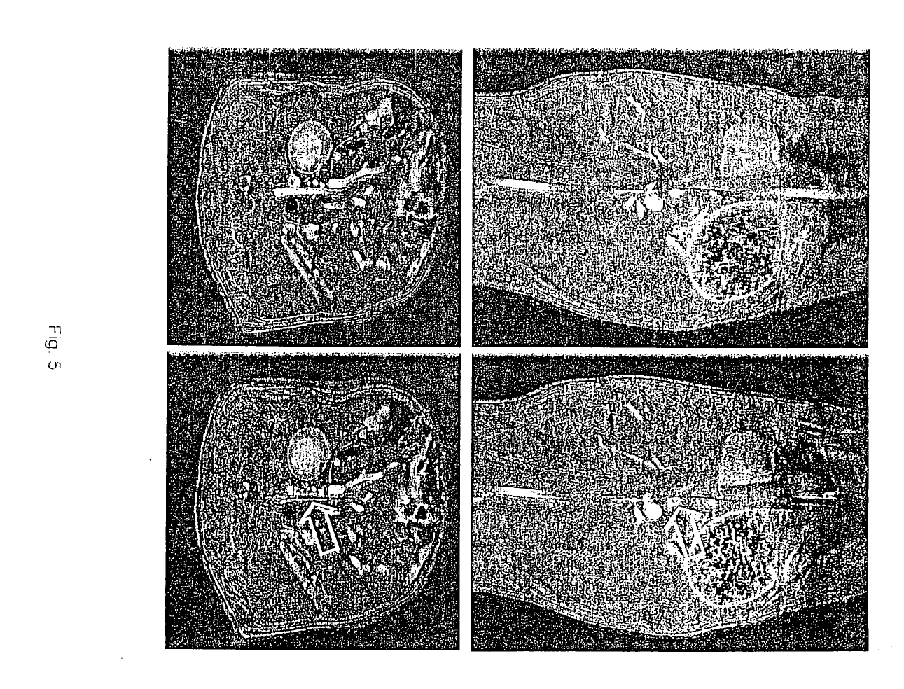

FIG. 5 shows high resolution gradient echo MR pictures of a Watanabe rabbit 12 hours after administration of a plaque-passable MR contrast medium with and without signal suppression of moving signal carriers in all three directions in space;

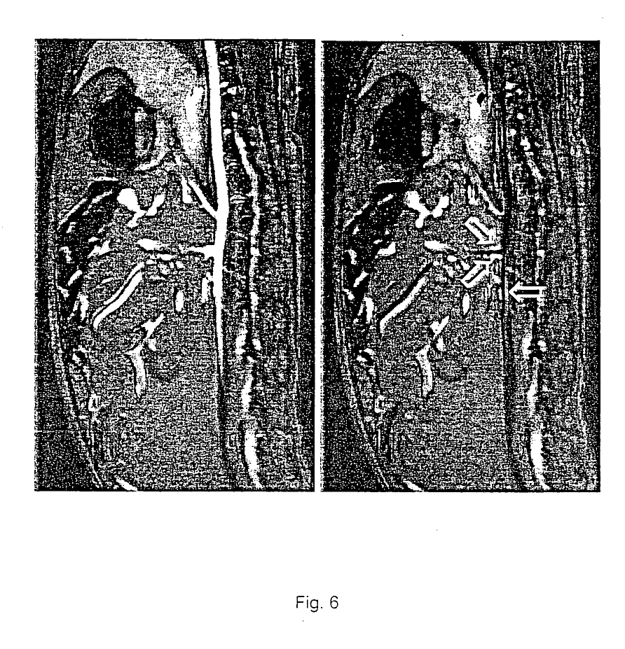

FIG. 6 shows high resolution gradient echo MR images as in FIG. 5, 28 hours after administering the plaque-passable MR contrast medium.

FIG. 1 shows first a diagrammatic visualization of a gradient echo pulse sequence for illustration of the parameters G_{bipolar}, tᵣₐₘₚ, tₚₗₐₜₑₐᵤ and tₛₑₚ in a plot of G(t) (gradient field intensity) over time t. The meaning of the individual parameters is explained in more detail above.

FIG. 2 reproduces gradient echo pulse sequences for imaging.

FIG. 2a shows first a sequence that has no flow dephasing gradient pulses for dephasing of moving nuclear spins, but rather a sequence with flow compensation, i.e. a sequence in which M₀ and M₁ are each zero. The figure shows the gradient switchings in the three direction in space over time.

In the top representation (“G_{slice}”) the gradient pulse sequence for slice selection in z-direction is shown. An excitation-RF pulse is emitted during the first gradient pulse. A certain slice is chosen by this slice gradient pulse since the corresponding resonance condition is only attained there. The following pulses in z-direction with the reverse or same polarity are used for repeated refocussing of the defocussing that has been caused by the first pulse and for setting the condition that both M₀ and also M₁ become zero.

In the bottom representation (“Gₚₕₐₛₑ”) pulses for phase coding of the nuclear spins are shown diagrammatically. With each repetition of the indicated pulse sequence, the size and polarity of this phase-gradient pulse are changed incrementally between two extreme values −Gₚₕₐₛₑ and +G_{phase.}

In the middle representation (“G_{read}”), the readout gradient pulses are reproduced. The pulse sequence, as in the case of the slice gradient pulses, is computed such that the conditions of M₀=0 and M₁=0 are satisfied. The nuclear spins, depending on their respective location, are frequency-encoded by the readout gradient pulses. During the last pulse, the signal of the nuclear spins in the x-y plane are formed by refocussing, which signal is recorded.

FIG. 2b reproduces a diagrammatic visualization of the gradient echo pulse sequence for recording two-dimensional MR data in a first embodiment according to the invention. This sequence contains, on the one hand, the sequence from FIG. 2a that does not have any flow dephasing gradient pulse sequences, but rather solely flow-compensated imaging gradient echo pulse sequences. In addition, gradient switchings are shown, labeled dark, that in addition have been inserted into the imaging sequences and that are used for dephasing the nuclear spins in moving media without the imaging gradient echo pulse sequences being influenced. In this case, the gradient moments of the first order M₁ were inserted into all directions in space (slice, phase and readout), such that suppression of the signals of the nuclear spins that move during the measurement in any direction in space takes place.

FIG. 2c reproduces a diagrammatic visualization of the gradient echo pulse sequence for recording two-dimensional MR data in a second embodiment according to the invention. In this sequence, the imaging gradient echo pulse sequences shown originally in FIG. 2a are no longer separately detectable. These gradient echo pulse sequences are formed by recomputation with consideration of the flow dephasing gradient pulse sequences.

The examples described below were implemented on clinical MR tomographs.

### EXAMPLE 1

In a first variant, the inserted bipolar gradient echo pulse sequences on a 1.5 tesla whole body tomograph (Magnetom Vision, Siemens, Erlangen) were used with a maximum gradient field intensity Gₘₐₓ=25 mT/m and a maximum slew rate sₘₐₓ=42 T/(m·s).

The initial sequence was a 3D-FLASH sequence whose parameter was optimized for imaging of small animals (high spatial resolution).

In order to demonstrate the effectiveness of suppression of the signal from the blood vessels, a contrast medium was administered to a Copenhagen rat with stimulated lymph nodes in an animal test. The contrast medium was selected such that it remained in the bloodstream for a long time and massively shortened the relaxation times T₁ and T₂ there. In this case, it was a gadolinium complex with a fluorinated side chain that had the following chemical composition: [10-{(RS)-1-[({[(5S)-6-{4-[(heptadecafluorooctyl)sulfonyl]piperazin-1-yl}-5-{[(alpha-D-mannopyranos-1-O-yl)oxy]acetylamino}-6-oxohexan-1-yl]carbamoyl}methyl)carbamoyl-kappa O]ethyl}-1,4,7,10-tetraazacyclododecane-1,4,7-triacetato(3)-kappa N1, kappa N4, kappa N7, kappa N10, kappa O1, kappa O4, kappa O7]-gadolinium. 50 μmol of Gd/kg of body weight was injected i.v.

In a first experiment, selective bipolar flow dephasing gradient pulse sequences were first inserted. The resulting pictures are shown in FIG. 3:

The conditions for pictures were as follows: echo time TE=14.0 ms, size of the field of view (FOV)=60×120 mm²; slice thickness SL=0.32 mm; matrix 104×256; BW=150 Hz/pixel; flip angle α=15°; recording time TA=3 minutes 42 s.

In the upper picture in FIG. 3, the inguinal lymph nodes of the rat are easily visible due to powerful contrast medium concentration (arrows). It can be recognized that blood vessels that run in the direction of the inserted gradient pulses are shown with little or no signals. Since the contrast medium that was used was taken up by the lymphatic system in which the moving speed of the signal carriers is very low compared to the blood stream, signal-rich imaging of the lymph nodes was achieved.

In the lower picture in FIG. 3, it can be furthermore recognized that the aorta (open arrows) that runs in the readout direction does not show any signal due to suppression in the readout direction. In contrast to this, the renal vein (closed arrow) that runs perpendicular to it was not suppressed, since a corresponding flow dephasing gradient pulse sequence was not switched in this direction. In any case, the signal from the renal veins could also clearly have been reduced if suitable flow dephasing gradient pulse sequences had been switched in addition in this direction. To do this, relatively large gradient moments of the first order M₁ would be necessary, since the velocity of the signal carrier in the veins is relatively low, so that it would have been necessary to reduce vₘₐₓ.

FIG. 4 shows pictures recorded with a 3D gradient echo pulse sequence with and without the flow dephasing gradient switchings compared to one another, in turned recorded on a Copenhagen rat. In this case, bipolar gradient pulses were inserted into all three directions in space. The corresponding pictures are shown on the right side of FIG. 4. On the left side, pictures are reproduced that were obtained without the influence of the flow dephasing gradient switchings.

In this test, the motion sensitivity vₘₐₓ was changed in the range from 2.56 cm/s to 36.5 cm/s by variation of the echo time TE from TEₘᵢₙ=9.4 ms to 18 ms. The other parameters were: G_{bipolar}=20 mT/m; tᵣₐₘₚ=0.6 ms; tₚₗₐₜₑₐᵤ=½(TE-TEₘᵢₙ)=0 to 8.6 ms; tₛₑₚ=3.7 ms; TR=19.1 ms to 25.5 ms; size of the field of view 40×80 mm²; slice thickness SL=0.5 mm; matrix: 128×256; BW=150 Hz/pixel; flip angle α=25°; recording time TA=2 minutes 29 seconds.

The pulse sequence was implemented such that with increasing echo time TE (from top to bottom in the picture sequence), smaller and smaller speeds were sufficient to suppress the signal of moving spins.

Since a prolonged echo time TE via intensified T₂ decay also causes a reduction of the MR signal, the experiment was also carried out for all echo times even without a conventional gradient for flow dephasing.

The figure shows a comparison of the two series of pictures that shows how the MR signal was increasingly suppressed in the aorta with decreasing vₘₐₓ such that the iliac lymph nodes next to the aorta could be distinguished better and better from the aorta. Clear differentiation from the aorta succeeds only below 10 cm/s.

### EXAMPLE 2

A second variant was implemented on a 1.5 tesla whole body tomograph (Magnetom Symphony, Siemens, Erlangen) with a maximum gradient field intensity Gₘₐₓ=30 mT/m and a maximum slew rate sₘₐₓ=120 T/(m·s).

In an animal experiment, a Watanabe rabbit was injected i.v. with the aforementioned intravascular gadolinium contrast medium [10-{(RS)-1-[({[(5S)-6-{4-[(heptadecafluorooctyl)sulfonyl]piperazin-1-yl}-5-{[(alpha-D-mannopyranos-1-O-yl)oxy]acetylamino}-6-oxohexan-1-yl]carbamoyl}methyl)carbamoyl-kappa O]ethyl}-1,4,7,10-tetraazacyclododecane-1,4,7-triacetato(3)-kappa N1, kappa N4, kappa N7, kappa N10, kappa O1, kappa O4, kappa O7]-gadolinium in an amount of 0.1 mmol/kg of body weight, which accumulates in plaques. At a given velocity sensitivity of vₘₐₓ=10 cm/s, echo times of between 8.0 ms and 9.5 ms were produced depending on the spatial resolution. Image data were recorded with and without flow dephasing in all three directions in space for an extended time after administration.

FIG. 5 shows image data that were obtained 12 hours after administration and FIG. 6 shows image data that were acquired 28 hours after administering the contrast medium.

Even after 28 hours the contrast medium signal in the blood vessel was still so strong that plaques could only be clearly identified only in the flow-dephased image data.

The pictures shown in FIG. 5 were recorded under the following conditions: TR=16 ms; TE=9.4 ms; FOV=135×180 mm²; SL=2 mm; matrix: 307×512; BW=245 Hz/pixel; α=30°; TA=2 minutes 37 seconds.

In this figure, high resolution gradient echo MR pictures without (pictures on the left) or with (pictures on the right) signal suppression of moving signal carriers are shown. While with flow dephasing the interior of the blood vessels (arrows) could be offset darkly against the plaque that appears bright and that takes up the contrast medium, in the picture without flow dephasing it was not possible to identify plaques.

For the picture in FIG. 6, the following parameters were used: TR=14 ms; TE=8.5 ms; FOV=200×200; SL=2 mm; matrix: 205×256; BW=245 Hz/pixel; α=30; TA=1 minute 32 seconds.

This figure shows pictures of the rabbit 28 hours after administering the plaque-passable gadolinium complex, the picture on the left having been obtained without and the one on the right with signal suppression of the moving signal carriers in all three directions in space (vₘₐₓ=10 cm/s). As in FIG. 5, only in the flow dephased measurement can the plaques (arrows) be distinguished from the blood vessels.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever. Also, any preceding examples can be repeated with similar success by substituting the generically or specifically described reactants and/or operating conditions of this invention for those used in such examples.

Throughout the specification and claims, all temperatures are set forth uncorrected in degrees Celsius and, all parts and percentages are by weight, unless otherwise indicated.

The entire disclosures of all applications, patents and publications, cited herein and of corresponding German application No. 102 60 372.3, filed Dec. 13, 2002, and U.S. Provisional Application Serial No. 60/446,563, filed Feb. 12, 2003 are incorporated by reference herein.

From the foregoing description, one skilled in the art can easily ascertain the essential characteristics of this invention and, without departing from the spirit and scope thereof, can make various changes and modifications of the invention to adapt it to various usages and conditions.

1. Imaging arrangement, comprising
a) a nuclear spin tomography device to obtain data for locally-resolved imaging of the magnetic resonance behavior of the atomic nuclei in a selected field of view in a body, the device being made and programmed such that the body can be exposed by the device to high frequency and magnetic field gradient echo pulse sequences that produce magnetization in a body such that the magnetization of a medium that is flowing in at least one direction in space in the body can be attenuated by dephasing the spins of the atomic nuclei in the medium, and
b) an MR contrast medium that is taken up by the body.

2. Arrangement according to claim 1, characterized in that the magnetization of the medium flowing in at least one direction in space in the body can be attenuated by dephasing of the spins by gradient moments of order i Mᵢ(t) being maximized in this direction in space according to the following relation:
$${M}_{i} ( t ) = γ \cdot {\int }_{0}^{t} G ( {t}^{′} ) \cdot {t}^{′ i } ⅆ {t}^{′}$$
whereby
i is an integer greater than zero,
γ is the gyromagnetic ratio of the atomic nuclei,
G(t′) is a time-dependent gradient field intensity in this direction in space and
t is the time interval that has passed since the emission of a high frequency pulse for excitation of the atomic nuclei.

3. Arrangement according to claim 2, wherein the magnetization of the medium flowing in at least one direction in space in the body can be attenuated by dephasing of the spins in that gradient moments of the first order M₁(t) are maximized in this direction in space according to the following relation:
$${M}_{1} ( t ) = γ \cdot {\int }_{0}^{t} G ( {t}^{′} ) \cdot {t}^{′ } ⅆ {t}^{′}$$

4. Arrangement according to one of the preceding claims, wherein gradient echo pulse sequences can be produced in the respective directions in space by inserting the flow dephasing gradient pulses into flow-compensated imaging gradient echo pulse sequences.

5. Arrangement according to claim 4, wherein M₁ satisfies the following relation:
M₁(t; G_{bipolar}, tᵣₐₘₚ, tₚₗₐₜₑₐᵤ, tₛₑₚ)=γ·G_{bipolar}·(tᵣₐₘₚ+tₚₗₐₜₑₐᵤ)·(2^{tramp}+tₚₗₐₜₑₐᵤ+tₛₑₚ) [7]

6. Arrangement according to one of the preceding claims, wherein the device
a static magnet,
gradient devices for producing gradient pulses in three directions in space that are orthogonal to one another,
a transmission device for producing high frequency signals,
a receiving device for high frequency signals,
a device for triggering gradient devices and the transmission device,
an evaluation device, and
a display device [sic].

7. Arrangement according to one of the preceding claims, wherein the MR contrast medium can be administered intravenously to a human or animal body.

8. Arrangement according to one of the preceding claims, wherein the MR contrast medium is lymph-passable and/or plaque-passable.

9. Process for locally-resolved imaging of the magnetic resonance behavior of atomic nuclei in a selected field of view in a body in which data from the field of view are obtained by means of a nuclear spin tomography device by the body being exposed to high frequency and magnetic field gradient echo pulse sequences that produce magnetization in the body such that the magnetization of a medium flowing in at least one direction in space is attenuated in the body by dephasing of the spins of the atomic nuclei in the medium and by an MR contrast medium being supplied to the body.

10. Process according to claim 9, wherein the magnetization of the medium flowing in at least one direction in space in the body is attenuated by dephasing of the spins by the gradient moments of order i Mᵢ(t) being maximized in this direction in space according to the following relation:
$${M}_{i} ( t ) = γ \cdot {\int }_{0}^{t} G ( {t}^{′} ) \cdot {t}^{′ i} ⅆ {t}^{′}$$
whereby
i is an integer greater than zero,
γ is the gyromagnetic ratio of the atomic nuclei,
G(t′) is a time-dependent gradient field intensity in this direction in space and
t is the time interval that has passed since the emission of a high frequency pulse for excitation of the atomic nuclei.

11. Process according to claim 10, wherein the magnetization of the medium flowing in at least one direction in space in the body is attenuated by dephasing of the spins by the gradient moments of the first order M₁(t) being maximized in this direction in space according to the following relation:
$${M}_{1} ( t ) = γ \cdot {\int }_{0}^{t} G ( {t}^{′} ) \cdot {t}^{′ } ⅆ {t}^{′}$$

12. Process according to one of claims 9-11, wherein gradient echo pulse sequences are produced in the respective directions in space by inserting the flow dephasing gradient pulses into flow-compensated imaging gradient echo pulse sequences.

13. Process according to claim 12, wherein M₁ satisfies the following relation:
M₁(t; G_{bipolar}, tᵣₐₘₚ, tₚₗₐₜₑₐᵤ, tₛₑₚ)=γG_{bipolar}·(tᵣₐₘₚ+tₚₗₐₜₑₐᵤ)·(2tᵣₐₘₚ+tₚₗₐₜₑₐᵤ+tₛₑₚ) [7]

14. Process according to one of claims 9-13, wherein the MR contrast medium is administered intravenously to a human or animal body.

15. Process according to one of claims 9-14, wherein the MR contrast medium is lymph-passable and/or plaque-passable.
